(19) United States
(12) Patent Application Publication
(10) Pub. No.: US 2026/0306809 A1
(43) Pub. Date: Oct. 8, 2026

(54) SLEEP BREATHING ASSIST DEVICE

(71) Applicant: YOU-RUEI LIN, Kaohsiung (TW)

(72) Inventors: YOU-RUEI LIN, Kaohsiung (TW); ZHENG-JEI LIN, Kaohsiung (TW)

(21) Appl. No.: 19/635,965

(22) Filed: Apr. 1, 2026

(30) Foreign Application Priority Data
Apr. 2, 2025 (TW) 114112904

(51) Int. Cl. A61M 29/00; A61F 5/56

(52) U.S. Cl. CPC A61M 29/00; A61F 5/56; A61M 2210/0618

(57) ABSTRACT

The present invention discloses a sleep breathing assist device comprising: an internal carrier comprising: a first straight tube, a second straight tube, a bending tube, a first curve part, and a second curve part, wherein the first straight tube is connected to the bending tube, the first curve part is connected to the first straight tube relative to the bending tube, wherein at least one first groove is formed on the first curve part so as to define a first air guiding space; the second straight tube is connected to the bending tube, and the second curve part is connected to the second straight tube relative to the bending tube, wherein at least one second groove is formed on the second curve part so as to define a second air guiding space; and an external coating layer, which covers an outer edge of the internal carrier.

### CROSS-REFERENCE TO RELATED APPLICATION

This application claims the benefit of Taiwan Patent Application No. 114112904, filed Apr. 2, 2025. The disclosure of the above application is incorporated herein by reference in its entirety.

### FIELD OF THE INVENTION

The present invention relates to a medical assist tool, and particularly pertains to a sleep breathing assist device able to enter laryngopharynx through nasal cavity so as to keep airway unobstructed.

### BACKGROUND OF THE INVENTION

A snore stopper refers to a device for smoothing airway or preventing blockage in a respiratory tract so as to relieve or to stop snoring. A nasal dilator, one type of common snore stoppers, reduces obstruction in a nasal tract by mechanically expanding the nasal cavity so that respiration through the nose is smoothed. Nasal sticker, another type of common snore stoppers, keeps the airway unobstructed by expanding the nasal cavity through its elasticity and tensile forces when sticked on the nose bridge.

Though the aforementioned snore stoppers ensure air inhalation amount by expanding the nasal cavity, airways of the patients with snoring symptoms remain obstructed during sleeping as soft palate contacts with upper laryngopharynx. Therefore, the airway at laryngopharynx remains closed, resulting in unsmooth airflow even with expanded nasal cavity. Accordingly, to address the above issues, a sleep breathing device capable of preventing the soft palate from contacting the laryngopharynx to ensure smooth airflow in the respiratory tract is in a urgent need.

### SUMMARY OF THE INVENTION

The object of the present invention is to provide a sleep breathing device capable of preventing the soft palate from contacting the laryngopharynx so as to guarantee unobstructed airflow in the respiratory tract.

In light of this, the present invention provides a sleep breathing assist device comprising: an internal carrier comprising a first straight tube, a second straight tube, a bending tube, a first curve part, and a second curve part, wherein: an end of the first straight tube is connected to an end of the bending tube, and the first straight tube extends at a first direction from a first position where the first straight tube is connected to the bending tube; an end of the first curve part is connected to an end of the first straight tube relative to the bending tube, and the first curve part bends from a second position where the first curve part is connected to the first straight tube, and the first curve part bends along the first direction to a second direction, wherein at least one first groove is formed on the first curve part, and each of the at least one first groove extends at a direction from the second position to the other end of the first straight tube, and a first air guiding space is defined between the at least one first groove and the first curve part; an end of the second straight tube is connected to the other end of the bending tube, and the second straight tube extends at a third direction from a third position where the second straight tube is connected to the bending tube; and an end of the second curve part is connected to an end of the second straight tube relative to the bending tube, and the second curve part bends from a fourth position where the second curve part is connected to the second straight tube, and the second curve part bends along the third direction to a fourth direction, wherein at least one second groove is formed on the second curve part, and each of the at least one second groove extends at a direction from the fourth position to the other end of the second straight tube, and a second air guiding space is defined between the at least one second groove and the second curve part; and an external coating layer covered on an outer edge of the internal carrier, wherein the external coating layer does not completely occupy the first air guiding space, and does not completely occupy the second air guiding space.

Preferably, a material of the internal carrier comprises a polymer resin, and the polymer resin comprises polyethylene or polypropylene.

Preferably, wherein a material of the internal carrier comprises an elastic body, and the elastic body comprises silicone rubber.

Preferably, a material of the external coating layer comprises a hydrophilic material.

Preferably, the hydrophilic material comprises a hydrogel.

Preferably, the hydrophilic material comprises a silicone hydrogel.

Preferably, at a direction from the second position to the other end of the first curve part, an opening size of the first air guiding space is in a gradual reduction, or in a gradual expansion followed by a gradual reduction; and at a direction from the fourth position to the other end of the second curve part, an opening size of the second air guiding space is in a gradual reduction, or in a gradual expansion followed by a gradual reduction.

Preferably, at a direction from the second position to the other end of the first curve part, a cross-sectional area of the first curve part is in a gradual reduction, or in a gradual expansion followed by a gradual reduction; and at a direction from the fourth position to the other end of the second curve part, a cross-sectional area of the second curve part is in a gradual reduction, or in a gradual expansion followed by a gradual reduction.

Preferably, the sleep breathing assist device further comprises: a first strip structure, forming at the outer edge of the first straight tube, and forming a recess or a protrusion on the first straight tube; a second strip structure, forming at the outer edge of the second straight tube, and forming a recess or a protrusion on the second straight tube; and a third strip structure, forming at the outer edge of the bending tube, and forming a recess or a protrusion on the bending tube.

Preferably, the first strip structure, the second strip structure, or the third stripe structure is in a shape comprising grid lines, paralleled lines, or dots; the first strip structure, the second strip structure, or the third stripe structure is in an arrangement comprising a linear arrangement, a matrix arrangement, or a radial arrangement.

The technical effects achieved by the present invention lies in: the first curve part and the second curve part are elastic and in a curve shape so that the first curve part and the second curve part may appropriately bend. The intrinsic elasticity enables the first curve part and the second curve part to restore their curved shape in the laryngopharynx after progressing through the nostrils and the nasal cavity and arriving at the laryngopharynx. In this way, the first curve part and the second curve part extends towards one end of the soft palate according to the airway curvature in the laryngopharynx. Concretely, the first curve part and the second curve part may provide a solid support as they closely conform to the soft palate of the upper laryngopharynx, thereby creating a gap between the soft palate and the laryngopharynx. As such, when the air is inhaled through the nasal cavity and arrives at the laryngopharynx, the airflow is allowed to smoothly flow through the airway without any obstruction leading to breathing difficulty.

In terms of further avoiding the soft palate from closing the respiratory tract by covering the outer edges of the first curve part or the second curve part, a first groove is formed on the first curve part, and a second groove is formed on the first curve part. In this way, the airflow is able to pass through the airway via the first air guiding space or the second air guiding space even when the soft palate covers the outer edge of the external coating layer, thereby maintaining unobstructed inhalation and exhalation without leading to sleep apnea or breathing difficulty. Moreover, the external coating layer is made of hydrophilic materials, and exhibits a certain air permeability. Thus, there would be no foreign body sensation when the sleep breathing assist device progresses into the nasal cavity and the laryngopharynx, thereby enabling long-term wearing on the human body with comfort.

### BRIEF DESCRIPTION OF THE DRAWINGS

FIG. 1A is a three-dimensional diagram to illustrate structural features of the sleep breathing assist device;

FIG. 1B is a three-dimensional diagram in an exploded view to illustrate structural features of the internal carrier and the external coating layer;

FIG. 2 is an enlarged partial view illustrating the structural features of the internal carrier;

FIG. 3 is a view illustrating a state of use of the sleep breathing assist device; and

FIG. 4 is another view illustrating a state of use of the sleep breathing assist device.

### DETAILED DESCRIPTION OF THE INVENTION

To facilitate a clearer understanding of the above and/or other objects, advantages, and features of the present invention, preferred embodiments are set forth below with detailed descriptions.

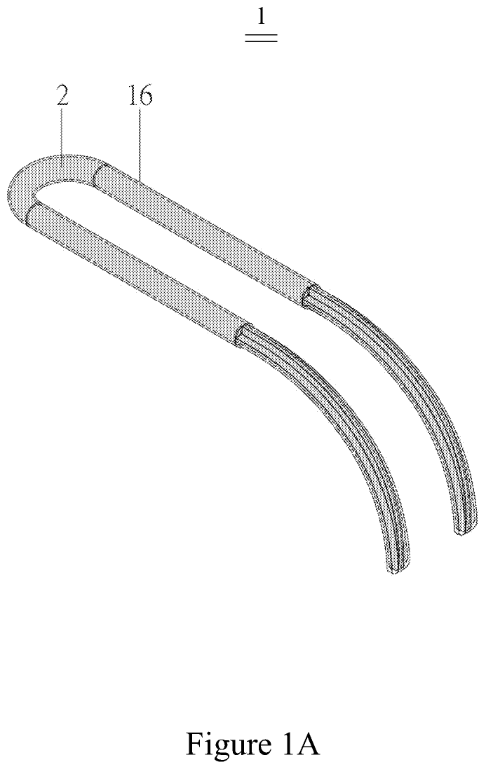

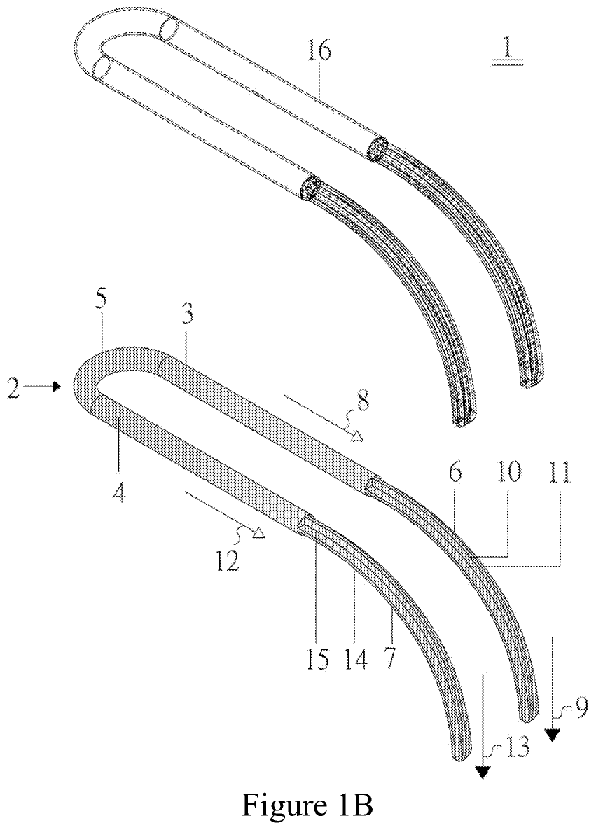

One object of the present invention aims to provide a sleep breathing assist device (1), as shown in FIGS. 1A to 1B, comprising: an internal carrier (2) and an external coating layer (16).

The internal carrier (2) comprises: a first straight tube (3), a second straight tube (4), a bending tube (5), a first curve part (6), and a second curve part (7), wherein: an first end of the first straight tube (3) is connected to an end of the bending tube (5), and the first straight tube (3) extends at a first direction (8) from a first position where the first straight tube (3) is connected to the bending tube (5); an end of the first curve part (6) is connected to an end of the first straight tube (3) relative to the bending tube (5), and the first curve part (6) bends from a second position where the first curve part (6) is connected to the first straight tube (3), and the first curve part (6) bends along the first direction (8) to a second direction (9), wherein at least one first groove (10) is formed on the first curve part (6), and each of the at least one first groove (10) extends at a direction from the second position to the other end of the first straight tube (3), and a first air guiding space (11) is defined between the at least one first groove (10) and the first curve part (6); an end of the second straight tube (4) is connected to the other end of the bending tube (5), and the second straight tube (4) extends at a third direction (12) from a third position where the second straight tube (4) is connected to the bending tube (5); and an end of the second curve part (7) is connected to an end of the second straight tube (4) relative to the bending tube (5), and the second curve part (7) bends from a fourth position where the second curve part (7) is connected to the second straight tube (4), and the second curve part (7) bends along the third direction (12) to a fourth direction (13), wherein at least one second groove (14) is formed on the second curve part (7), and each of the at least one second groove (14) extends at a direction from the fourth position to the other end of the second straight tube (4), and a second air guiding space (15) is defined between the at least one second groove (14) and the second curve part (7); and an external coating layer (16) covering an outer edge of the internal carrier (2), wherein the external coating layer (16) does not completely occupy the first air guiding space (11), and does not completely occupy the second air guiding space (15).

In preferred embodiments, the internal carrier (2) is bendable, and conformation of the internal carrier (2) can be restored once the internal carrier (2) is relieved from a bending state owing to intrinsic elasticity of the material. The material of the internal carrier (2) may be plastic, silicone rubber, or any elastic body, but not limited to this.

In some preferred embodiments, the thickness of the external coating layer (16) covering the internal carrier (2) is uniform and thin so that the airflow would not be obstructed in the first air guiding space (11), or the second air guiding space (15), thereby ensuring unobstructed airflow through the respiratory tract into a human body after passing through the nasal cavity into the laryngopharynx.

In other preferred embodiments, the first direction (8) is identical to the third direction (12), and the second direction (9) is identical to the fourth direction (13). In this way, the first straight tube (3), the bending tube (5) and the second straight tube (4) can form a U-shaped tube, and the first curve part (6) and the second curve part (7) exhibit the same bending direction and curvature.

Specifically, a method for using the sleep breathing device (1) comprises:
(S1) aligning the first curve part (6) and the second curve part (7) to the nostrils, respectively, and bringing the first curve part (6) and the second curve part (7) into proximity of the laryngopharynx via the nasal cavity, wherein the first curve part (6) and the second curve part (7) may be appropriately bent; and
(S2) slightly adjusting positions of the first curve part (6) and the second curve part (7) so that the first curve part (6) and the second curve part (7) are in contact with an end of the soft palate.

The first curve part (6) and the second curve part (7) holds the soft palate when in contact with the end of the soft palate, thereby creating a gap between the soft palate and the upper laryngopharynx. Moreover, the bending tube (5) limits how far the first curve part (6) and the second curve part (7) can move in the human body so as to prevent both of the first curve part (6) and the second curve part (7) from excessively entering the human body, thereby avoiding discomfort.

In various embodiments, in order to provide the internal carrier (2) elasticity, flexibility, structural intensity, endurance, or structural support, a material of the internal carrier comprises a polymer resin, and the polymer resin may be polyethylene or polypropylene, but not limited to this.

In various embodiments, in order to provide the internal carrier (2) elasticity, flexibility, structural intensity, endurance, or structural support, a material of the internal carrier comprises an elastic body, and the elastic body may be silicone rubber, but not limited to this.

In various embodiments, in order to provide air permeability so that wearing comfort may be improved while reducing foreign body sensation, a material of the external coating layer comprises a hydrophilic material, but not limited to this.

In various embodiments, the hydrophilic material may be a hydrogel, and the hydrogel may be selected from a group consisting of polyhydroxyethylmethacrylate (PHEMA), Nelfilcon A, Etafilcon A, Hilafilcon B, Oculfilcon B, Omafilcon A, and Polymacon, but not limited to this.

In various embodiments, the hydrophilic material may be a silicone hydrogel, and the silicone hydrogel may be selected from a group consisting of Balafilcon A, Lotrafilcon B, Comfilcon A, Galyfilcon A, and Narafilcon A, but not limited to this.

In some preferred embodiments, in order to make airflow unobstructed in the airway, and, at mean time, to minimize foreign body sensation and provide better comfortable wearing experience, a cross-sectional shape of the first groove (10) or the second groove (14) may be a V shape or a U shape, but not limited to this.

More preferably, at a direction from the second position to the other end of the first curve part (6), an opening size of the first air guiding space (11) is in a gradual reduction, or in a gradual expansion followed by a gradual reduction; and at a direction from the fourth position to the other end of the second curve part (7), an opening size of the second air guiding space (15) is in a gradual reduction, or in a gradual expansion followed by a gradual reduction, but not limited to this.

More preferably, at a direction from the second position to the other end of the first curve part (6), a cross-sectional area of the first curve part (6) is in a gradual reduction, or in a gradual expansion followed by a gradual reduction; and at a direction from the fourth position to the other end of the second curve part (7), a cross-sectional area of the second curve part (7) is in a gradual reduction, or in a gradual expansion followed by a gradual reduction, but not limited to this.

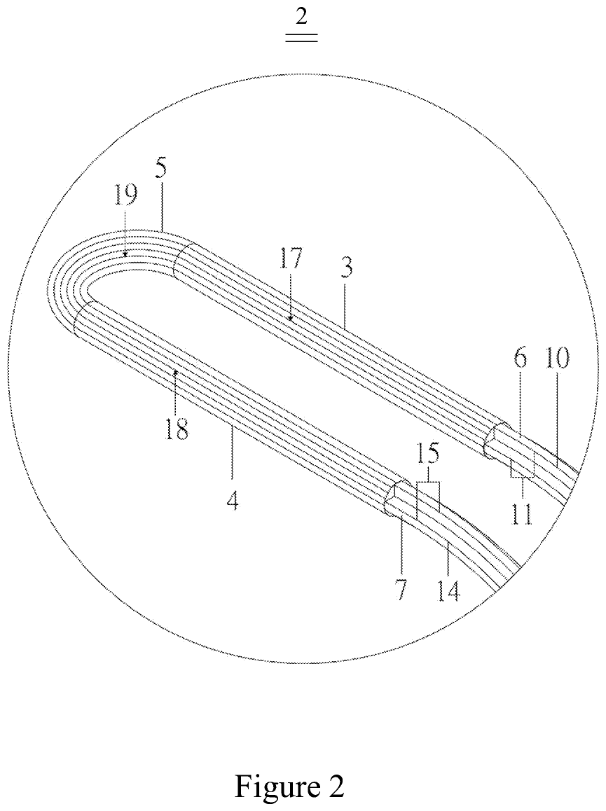

In other preferred embodiments, in order to increase the friction between the external coating layer (16) and the internal carrier (2) so that no relative movement between the external coating layer (16) and the internal carrier (2) would occur, as shown in FIG. 2, the sleep breathing assist device (1) further comprises: a first strip structure (17), forming at the outer edge of the first straight tube (3), and forming a recess or a protrusion on the first straight tube (3); a second strip structure (18), forming at the outer edge of the second straight tube (4), and forming a recess or a protrusion on the second straight tube (4); and a third strip structure (19), forming at the outer edge of the bending tube (5), and forming a recess or a protrusion on the bending tube (5).

More preferably, the first strip structure (17), the second strip structure (18), or the third stripe structure (19) may be in a shape including grid lines, paralleled lines, or dots, but not limited to this. On the other hand, the first strip structure (17), the second strip structure (18), or the third stripe structure (19) may be in an arrangement including a linear arrangement, a matrix arrangement, or a radial arrangement, but not limited to this.

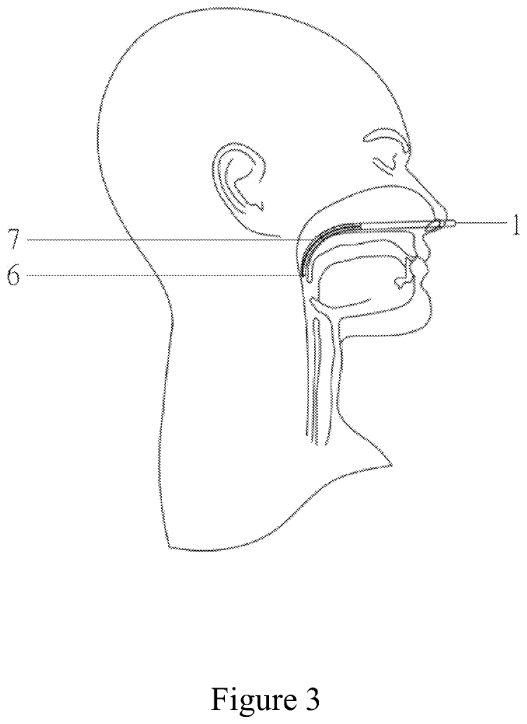

As shown in FIG. 3, the first curve part (6) and the second curve part (7) contacts with the soft palate when the sleep breathing assist device (1) reaches the laryngopharynx via the nostril and the nasal cavity, and a gap is created between the soft palate and the laryngopharynx.

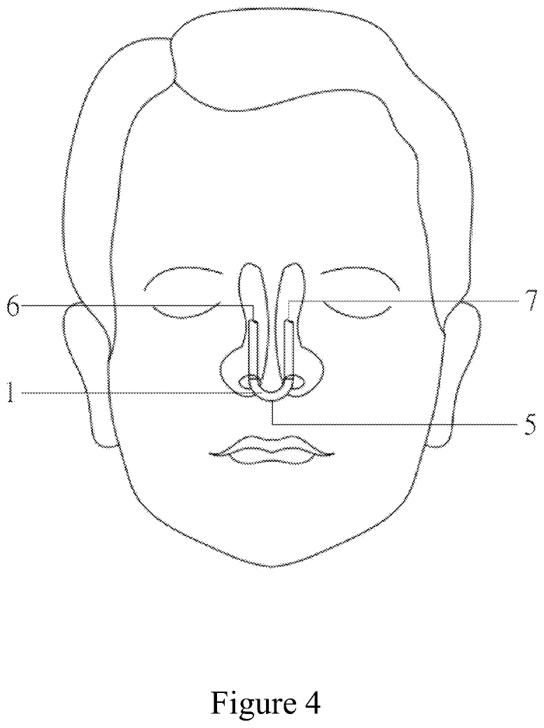

As shown in FIG. 4, clearly, the bending tube (5) provides a limiting structure so that excessive entry of the first curve part (6) and the second curve part (7) into the human is avoided, thereby ensuring comfortable wearing experience.

The present invention achieves the following advantageous technical effects:

The first curve part (6) and the second curve part (7), owing to their intrinsic elasticity and bent shape, can reach the end of soft palate along the curvature of airway in the laryngopharynx. Also, the intrinsic elasticity and the bent shape enable the first curve part (6) and the second curve part (7) to be appropriately bent, and their bent shape can be restored after they arrive at the laryngopharynx via the nostrils and nasal cavity.

In particular, the first curve part (6) and the second curve part (7) can conform with the soft palate in the upper laryngopharynx so as to provide support. In this manner, a gap between the soft palate and the upper laryngopharynx can be created, allowing unobstructed airflow along the airway once the air is inhaled from the nasal cavity to the laryngopharynx, thereby preventing airway obstruction and breathing difficulty.

On the other hand, to prevent the soft palate from covering the outer edge of the first curve part (6) or the second curve part (7), which may close the airway, a first groove (10) is formed on the first curve part (6) and a second groove (14) is formed on the second curve part (7). Therefore, airflow remains smooth in the first air guiding space (11) or the second air guiding space (15) even when the soft palate conforms to the external coating layer (16). As such, inhalation and exhalation are not blocked, thereby avoiding sleep apnea or breathing difficulty.

Furthermore, the sleep breathing assist device (1) does not lead to any foreign body sensation when entering the nasal cavity and the laryngopharynx as the external coating layer (16) is made of hydrophilic materials and exhibits air permeability, thereby allowing long-term body wearing with comfortable wearing experience.

The foregoing descriptions merely illustrate preferred embodiments of the present invention and are not intended to limit the scope of implementation thereof. Any simple equivalent modifications or alterations made based on the claims and the disclosure of the present invention shall fall within the scope of the present invention. In addition, no embodiment or claim of the present invention is required to achieve all of the objectives, advantages, or features disclosed herein. Furthermore, the abstract and the title are provided solely to facilitate patent searching and shall not be construed as limiting the scope of the present invention. Moreover, the terms “first,” “second,” and the like, as used in the specification, are merely employed to distinguish components by name and shall not be interpreted as imposing numerical limitations on the quantity of such components.

What is claimed is:

1. A sleep breathing assist device comprising:
an internal carrier comprising a first straight tube, a second straight tube, a bending tube, a first curve part, and a second curve part, wherein:
an end of the first straight tube is connected to an end of the bending tube, and the first straight tube extends at a first direction from a first position where the first straight tube is connected to the bending tube;
an end of the first curve part is connected to an end of the first straight tube relative to the bending tube, and the first curve part bends from a second position where the first curve part is connected to the first straight tube, and the first curve part bends along the first direction to a second direction, wherein at least one first groove is formed on the first curve part, and each of the at least one first groove extends at a direction from the second position to the other end of the first straight tube, and a first air guiding space is defined between the at least one first groove and the first curve part;
an end of the second straight tube is connected to the other end of the bending tube, and the second straight tube extends at a third direction from a third position where the second straight tube is connected to the bending tube; and
an end of the second curve part is connected to an end of the second straight tube relative to the bending tube, and the second curve part bends from a fourth position where the second curve part is connected to the second straight tube, and the second curve part bends along the third direction to a fourth direction, wherein at least one second groove is formed on the second curve part, and each of the at least one second groove extends at a direction from the fourth position to the other end of the second straight tube, and a second air guiding space is defined between the at least one second groove and the second curve part; and
an external coating layer covering an outer edge of the internal carrier, wherein the external coating layer does not completely occupy the first air guiding space, and does not completely occupy the second air guiding space.

2. The sleep breathing assist device according to claim 1, wherein a material of the internal carrier comprises a polymer resin, and the polymer resin comprises polyethylene or polypropylene.

3. The sleep breathing assist device according to claim 1, wherein a material of the internal carrier comprises an elastic body, and the elastic body comprises silicone rubber.

4. The sleep breathing assist device according to claim 1, wherein a material of the external coating layer comprises a hydrophilic material.

5. The sleep breathing assist device according to claim 4, wherein the hydrophilic material comprises a hydrogel.

6. The sleep breathing assist device according to claim 4, wherein the hydrophilic material comprises a silicone hydrogel.

7. The sleep breathing assist device according to claim 1, wherein:
at a direction from the second position to the other end of the first curve part, an opening size of the first air guiding space is in a gradual reduction, or in a gradual expansion followed by a gradual reduction; and
at a direction from the fourth position to the other end of the second curve part, an opening size of the second air guiding space is in a gradual reduction, or in a gradual expansion followed by a gradual reduction.

8. The sleep breathing assist device according to claim 1, wherein:
at a direction from the second position to the other end of the first curve part, a cross-sectional area of the first curve part is in a gradual reduction, or in a gradual expansion followed by a gradual reduction; and
at a direction from the fourth position to the other end of the second curve part, a cross-sectional area of the second curve part is in a gradual reduction, or in a gradual expansion followed by a gradual reduction.

9. The sleep breathing assist device according to claim 1, wherein the sleep breathing assist device further comprises:
a first strip structure, forming at the outer edge of the first straight tube, and forming a recess or a protrusion on the first straight tube;
a second strip structure, forming at the outer edge of the second straight tube, and forming a recess or a protrusion on the second straight tube; and
a third strip structure, forming at the outer edge of the bending tube, and forming a recess or a protrusion on the bending tube.

10. The sleep breathing assist device according to claim 9, wherein:
the first strip structure, the second strip structure, or the third stripe structure is in a shape comprising grid lines, paralleled lines, or dots;
the first strip structure, the second strip structure, or the third stripe structure is in an arrangement comprising a linear arrangement, a matrix arrangement, or a radial arrangement.
